(19) United States
(12) Patent Application Publication
(10) Pub. No.: US 2026/0298799 A1
(43) Pub. Date: Oct. 1, 2026

(54) METHOD FOR EVALUATING CORROSION RESISTANCE OF ALUMINUM SHEET

(71) Applicant: HONDA MOTOR CO., LTD., Tokyo (JP)

(72) Inventors: Yuya KOTANI, Tokyo (JP); Daichi ASARI, Wako-shi (JP)

(21) Appl. No.: 19/573,416

(22) Filed: Mar. 20, 2026

(30) Foreign Application Priority Data
Mar. 27, 2025 (JP) 2025-053354

(51) Int. Cl. G01N 17/00; G01N 17/02; G01N 33/208; G01Q 60/32

(52) U.S. Cl. CPC G01N 17/006; G01N 17/02; G01Q 60/32; G01N 33/208

(57) ABSTRACT

A method for evaluating corrosion resistance of an aluminum sheet includes: preparing an aluminum sheet containing dissimilar metal particles in a matrix metal; creating an observation surface by treating a surface of the aluminum sheet with hydrochloric acid; observing a potential difference between the dissimilar metal particles and their surroundings by using an observation device; and evaluating corrosion resistance of the aluminum sheet from a product of the potential difference and the number of the dissimilar metal particles.

### CROSS REFERENCE TO RELATED APPLICATION

The entire disclosure of Japanese Patent Application No. 2025-053351, filed on Mar. 27, 2025, is incorporated herein by reference in its entirety.

### BACKGROUND

The present disclosure relates to a method for evaluating corrosion resistance of an aluminum sheet.

In general, in a metal product using a metallic material, the amount of a dissimilar metal added is adjusted to improve its functions such as strength and corrosion resistance. However, if the amount of the dissimilar metal added is increased, the amount of corrosion of the metallic material may increase. Furthermore, in a painted or plated metallic material, filiform rust may occur under the coating and filiform corrosion may progress. In a case of an aluminum sheet, it has been thought that precipitates in the aluminum sheet affects filiform corrosion. Meanwhile, performance verification has to be conducted to confirm how much corrosion will actually develop. Therefore, until now, it has not been possible to judge the corrosion resistance of an aluminum sheet without conducting long-term performance verification after the aluminum sheet is painted.

The above corrosion reaction is driven by local cells on a metallic material surface formed between a matrix metal and dissimilar metal particles or the like. For example, in the corrosion reaction of an aluminum sheet, at anodic spots on the surface of the aluminum sheet, electrons are generated as the underlying aluminum (Al) causes a dissolution reaction, whereas at cathodic spots, the electrons generated at the anodic spots cause a reduction reaction of oxidants such as water and hydrogen. Therefore, one of conceivable methods for evaluating the corrosion resistance of an aluminum sheet is to observe cathodic spots and anodic spots present on an aluminum sheet surface.

As a surface observation method of a metallic material containing dissimilar metal particles in a matrix metal, for example, Patent Literature 1 (JP7141701B) discloses a method for evaluating chemical conversion treatability. In this method, an observation surface with a maximum height difference of 200 nm or less created on a surface of a metallic material is observed using a Kelvin force microscope to measure a potential difference between the dissimilar metal particles and their surroundings, and the chemical conversion treatability of the metallic material is evaluated from this potential difference.

### SUMMARY

The surface observation method of a metallic material in Patent Literature 1 can evaluate the chemical conversion treatability of a metallic material but cannot evaluate the corrosion resistance.

An object of the present disclosure is to provide a method for evaluating corrosion resistance of an aluminum sheet, capable of evaluating the corrosion resistance of an aluminum sheet without performing a long-term corrosion resistance test.

As a result of earnest studies, the present inventors found the following matters.

A surface potential on a specimen, which was a surface of an aluminum sheet that had been mirror-polished and then surface-treated with hydrochloric acid, was measured using a Kelvin probe force microscope. Then, it was found that the amount of corrosion could be predicted by multiplying the potential difference between the dissimilar metal particles and their surroundings and the number of the dissimilar metal particles.

In sum, in order to achieve the aforementioned object, a method for evaluating corrosion resistance of an aluminum sheet according to the present disclosure includes: preparing an aluminum sheet containing dissimilar metal particles in a matrix metal; treating a surface of the aluminum sheet with hydrochloric acid to create an observation surface; observing a potential difference between the dissimilar metal particles and their surroundings using an observation device; and evaluating the corrosion resistance of the aluminum sheet from a product of the potential difference and the number of the dissimilar metal particles.

According to the method for evaluating corrosion resistance of an aluminum sheet in the present disclosure, it is possible to evaluate the corrosion resistance of an aluminum sheet without performing a long-term corrosion resistance test.

### BRIEF DESCRIPTION OF THE ACCOMPANYING DRAWINGS

FIG. 1 is a flowchart showing a method for evaluating corrosion resistance of an aluminum sheet in an embodiment.

FIG. 2 is a graph showing a relationship between Et of matrix precipitates and the area of filiform rust in Example.

FIG. 3 is a graph showing a relationship between Et of an intermetallic compound and the area of filiform rust in Example.

### DESCRIPTION OF THE EMBODIMENTS

Hereinafter, an embodiment of the present disclosure will be described in detail, but embodiments of the present invention should not be limited to the following embodiment.

A method for evaluating corrosion resistance of an aluminum sheet is a method for evaluating corrosion resistance of an aluminum sheet containing dissimilar metal particles in a matrix metal. In the method for evaluating corrosion resistance of an aluminum sheet, an observation surface is created by treating a surface of an aluminum sheet with hydrochloric acid. Then, a potential difference between the dissimilar metal particles and their surroundings is observed and the corrosion resistance of the aluminum sheet is evaluated from the product of this potential difference and the number of the dissimilar metal particles.

Aluminum for use in the aluminum sheet is, for example, an Al—Mg—Si-based alloy, namely, a 6000 series alloy (for example, A6061, A6063, or A6N01). However, the aluminum is not limited to the 6000 series alloy but other series alloys may be used. Components in an aluminum sheet may be adjusted as appropriate according to a desired purpose.

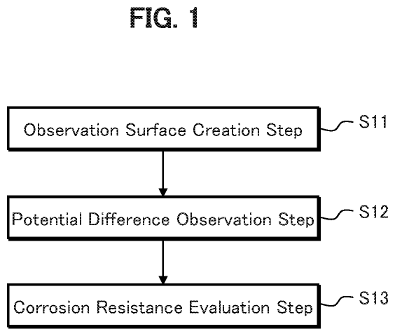

In the present embodiment, the method for evaluating corrosion resistance of an aluminum sheet will be described as having an observation surface creation step S11, a potential difference observation step S12, and a corrosion resistance evaluation step S13 as shown in FIG. 1.

### (Observation Surface Creation Step)

The observation surface creation step S11 is a step of creating an observation surface on a surface of an aluminum sheet. For creation of an observation surface, the surface of the aluminum sheet to serve as the observation surface is first mirror-polished. In the mirror polishing, the surface of the aluminum sheet is first polished with waterproof sandpaper, and then polished a diamond abrasive (for example, DP-paste). In this way, the surface with a maximum height difference of, for example, 180 nm or less is obtained. The maximum height difference of the surface of the aluminum sheet may be 80 nm or less or 30 nm or less. As will be described later, the maximum height difference of the observation surface of the aluminum sheet may be 200 nm or less. The surface treatment with hydrochloric acid may increase roughness of the surface of the aluminum sheet. Therefore, the observation surface may be adjusted such that the maximum height difference will be 200 nm or less after the surface treatment with hydrochloric acid.

The surface of the aluminum sheet may be mirror-polished by buffing with alumina particles, diamond particles, colloidal silica, or the like or by ion milling.

Next, the mirror-polished aluminum sheet is surface-treated with hydrochloric acid. As described above, in a corrosion reaction of an aluminum sheet, at anodic spots on the surface of the aluminum sheet, electrons are generated as the underlying aluminum (Al) causes a dissolution reaction. At cathodic spots on the same surface, the electrons generated at the anodic spots cause a reduction reaction of oxidants such as water and hydrogen. In the case of filiform corrosion of an aluminum sheet, this reaction is promoted under an acidic environment. Meanwhile, an aluminum sheet to be applied to an automobile undergoes a surface preparation called a zirconium chemical conversion treatment before painting. Since this surface preparation is performed under acidic conditions, acid-soluble dissimilar metal particles in the matrix metal are dissolved and detached during the surface preparation. Therefore, in order to evaluate the corrosion resistance of an aluminum sheet, it is essential to observe cathodic spots and anodic spots present on the surface of the aluminum sheet after the surface treatment with acid.

The surface treatment with hydrochloric acid can be carried out by immersing an aluminum sheet in, for example, 33% or more and 36% or less hydrochloric acid for 30 or more and 40 or less seconds.

The maximum height difference of the observation surface of the aluminum sheet may be 200 nm or less. When the maximum height difference of the observation surface of the aluminum sheet is 200 nm or less, a clear surface potential image is easily obtained in an observation of the observation surface with an observation device such as a Kelvin force microscope, as will be described later. This makes it easy to observe the potential difference between the dissimilar metal particles and their surroundings. The maximum height difference of the observation surface of the aluminum sheet may be 100 nm or less or 50 nm or less from the viewpoint of obtaining a clearer surface potential image and thereby making it easier to observe the potential difference between the dissimilar metal particles and their surroundings. The smaller the maximum height difference of the observation surface of the aluminum sheet, the better, and it is ideal to have no height difference at all. However, from the viewpoint of making it easy to prepare the observation surface, the lower limit may be set to, for example, 5 nm.

### (Potential Difference Observation Step)

The potential difference observation step S12 is a step of observing the potential difference between the dissimilar metal particles and their surroundings using an observation device. The observation device is, for example, a Kelvin force microscope. The Kelvin force microscope is a microscope capable of obtaining a surface potential image of a sample together with a surface shape of the sample. The Kelvin force microscope makes it possible to observe, as an image, how much voltage is applied to which region of the sample surface when a voltage is applied between the sample and a probe. Specifically, the Kelvin force microscope scans the sample surface with the probe, feeds back a voltage with which the amplitude of an electrostatic force component due to an excitation voltage goes to zero, and measures the potential on the sample surface using this feedback voltage, thereby forming a surface potential image.

The feedback voltage of the Kelvin force microscope is easily affected by a change in the distance between the sample surface and the probe. For this reason, if great roughness exists in the observation surface, the surface potential image is blurred, making it difficult to fully observe the potential difference between the dissimilar metal particles and their surroundings. However, when the maximum height difference of the observation surface is adjusted to, for example, 200 nm or less, the influence of the roughness of the observation surface on the feedback voltage can be reduced, making it easy to obtain a clear surface potential image. Then, the potential difference between the dissimilar metal particles and their surroundings on the observation surface of the aluminum sheet is observed with the Kelvin force microscope.

In the potential difference observation step S12, the number of the dissimilar metal particles on the observation surface of the aluminum sheet is counted by, for example, image analysis.

### (Corrosion Resistance Evaluation Step)

The corrosion resistance evaluation step S13 is a step of evaluating the corrosion resistance of the aluminum sheet from the product of the potential difference between the dissimilar metal particles and their surroundings and the number of the dissimilar metal particles.

In the corrosion resistance evaluation step S13, the product (Et) of the potential difference between the dissimilar metal particles and their surroundings and the number of the dissimilar metal particles is calculated. Et, which is the product of the potential difference and the number of the dissimilar metal particles, correlates to the occurrence of filiform rust. The higher the value Et, the more easily filiform rust will occur. Therefore, the amount of corrosion due to filiform rust in an aluminum sheet can be predicted based on the value Et, and accordingly the corrosion resistance (filiform corrosion resistance) of the aluminum sheet can be evaluated. Specifically, as will be described later, for example, the calculated value Et is checked against a graph created in advance for the same series of aluminum, so that the amount of corrosion due to filiform rust in the aluminum sheet can be predicted, and the corrosion resistance of the aluminum sheet can be evaluated. When two or more dissimilar metals are present as dissimilar metal particles, the particles of the dissimilar metal having the highest potential are used to obtain the potential difference between the dissimilar metal particles and their surroundings. The potential at the surroundings of the dissimilar metal particles is a potential at a portion on the observation surface where no dissimilar metal particles are generated.

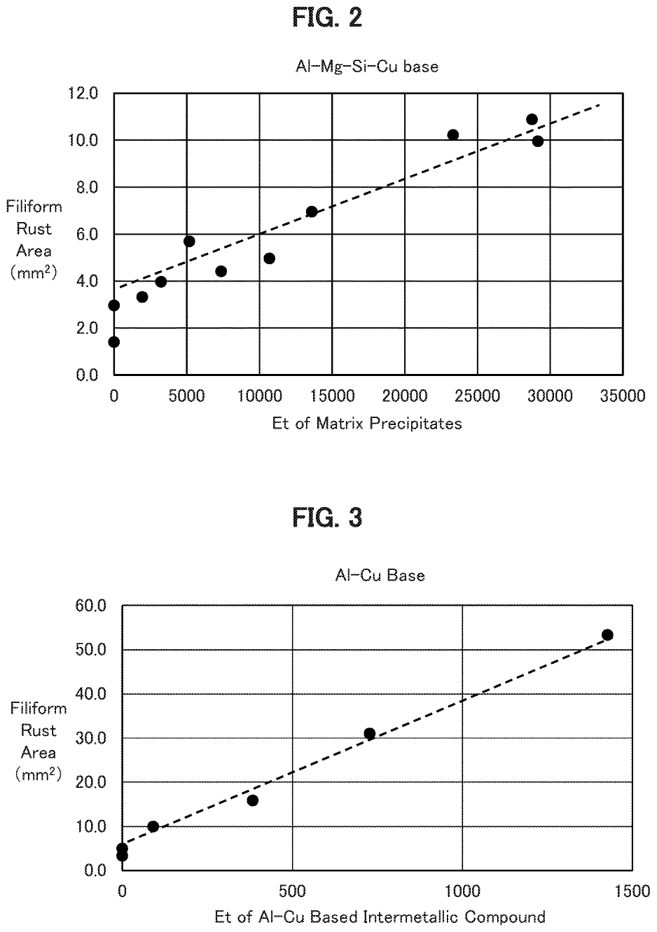

The dissimilar metal particles mean matrix precipitates, which are precipitates of the matrix of the aluminum sheet, and an intermetallic compound. As shown in FIGS. 2 and 3 to be described later, in a case where no Al—Cu-based intermetallic compound is generated, the occurrence of filiform rust correlates to matrix precipitates. On the other hand, in a case where an Al—Cu-based intermetallic compound is generated, the occurrence of filiform rust correlates to the intermetallic compound. Therefore, in the case where no Al—Cu-based intermetallic compound is generated, the number of matrix precipitates is set as the number of the dissimilar metal particles. In the case where the Al—Cu-based intermetallic compound is generated, the number of particles of the Al—Cu-based intermetallic compound is set as the number of the dissimilar metal particles.

In short, the dissimilar metal particles are at least matrix precipitates or particles of an Al—Cu-based intermetallic compound. If there is no Al—Cu-based intermetallic compound, the corrosion resistance of the aluminum sheet is evaluated based on the matrix precipitates. If there is an Al—Cu-based intermetallic compound, the corrosion resistance of the aluminum sheet is evaluated based on the Al—Cu-based intermetallic compound.

Specifically, if there is no Al—Cu-based intermetallic compound, the potential difference between the matrix precipitates and their surroundings is observed. The corrosion resistance of the aluminum sheet is evaluated from the product of this potential difference and the number of the matrix precipitates. On the other hand, if there is an Al—Cu-based intermetallic compound, the potential difference between particles of the Al—Cu-based intermetallic compound and their surroundings is observed. The corrosion resistance of the aluminum sheet is evaluated from the product of this potential difference and the number of the particles of the Al—Cu-based intermetallic compound.

### Examples

Hereinafter, the present invention will be described in detail by using Examples, but the present invention should not be limited to these Examples.

First, each aluminum sheet having a predetermined component composition was prepared.

Next, a surface of the aluminum sheet was polished by using waterproof sandpaper. The aluminum sheet was polished to a thickness of 3 μm using waterproof sandpaper with grit sizes of #120, #320, #600, and #1000 in that order. Next, the surface of the aluminum sheet was polished using a diamond abrasive (DP-paste) to a thickness of 0.25 μm. Subsequently, the aluminum sheet was surface-treated by being immersed in 36% hydrochloric acid for 30 seconds. In this way, the observation surface was created on the surface of the aluminum sheet.

Next, the observation surface of the aluminum sheet was observed with a Kelvin force microscope (manufactured by Hitachi High-Tech Science Corporation: multifunction scanning probe microscope unit AFM5200S). Then, the product (Et) of the number of matrix precipitates (generation amount) and the potential difference between the matrix precipitates and their surroundings (local potential difference) was calculated. The product (Et) of the number of particles of an intermetallic compound (generation amount) and the potential difference between the particles of the intermetallic compound and their surroundings (local potential difference) was calculated. The maximum height difference of the observation surface was measured using an atomic force microscope.

Next, a filiform rust test was carried out. In the filiform rust test, the observation surface of the aluminum sheet was sprayed with a 5% NaCl solution at 35° C., and was then left in an environment of 40° C. and 70% RH for 240 hours. This operation was repeated four times. Next, the area of filiform rust that had occurred was calculated by image analysis in which each portion bulged on the observation surface was determined to be a location where filiform rust had occurred.

Table 1 shows the components of aluminum sheets and their results. In addition, FIGS. 2 and 3 show a relationship between Et of matrix precipitates and the area of filiform rust and a relationship between Et of the intermetallic compound and the area of filiform rust.

TABLE 1

| | | | | | | | | | | Matrix Precipitate | | | Intermetallic | | | | |
|---|---|---|---|---|---|---|---|---|---|---|---|---|---|---|---|---|---|
| | | | | | | | | | | Al—Mg— | | | Compound | | | | |
| | | | | | | | | | | Si—Cu Base | | | Al—Cu Base | | | | Maxi- |
| | | | | | | | | | | | Local | | | Local | | Fili- | mum |
| | | | | | | | | | | Gener- | Po- | | Gener- | Po- | | form | Height |
| | | | | | | | | | | ation | tential | | ation | tential | | Rust | Differ- |
| ID | Chemical Composition (% by mass) | | | | | | | | | A- | Differ- | | A- | Differ- | | Area | ence |
| No. | Si | Fe | Cu | Mn | Mg | Cr | Zn | Ti | Ni | mount | ence | Eₜ | mount | ence | Eₜ | (mm²) | (nm) |
| 1 | 0.99 | 0.18 | 0.26 | 0.06 | 0.63 | 0 | 0.11 | 0 | 0 | 648 | 21 | 13608 | 0 | 0 | 0 | 7.0 | 46 |
| 2 | 0.98 | 0.39 | 0.3 | 0.53 | 0.6 | 0 | 0.11 | 0 | 0 | 405 | 8 | 3240 | 0 | 0 | 0 | 4.0 | 83 |
| 3 | 1 | 0.39 | 0.3 | 1.04 | 0.59 | 0 | 0.11 | 0 | 0 | 0 | 0 | 0 | 0 | 0 | 0 | 3.0 | 170 |
| 4 | 1 | 0.41 | 0.27 | 0.06 | 2.23 | 0 | 0.12 | 0 | 0 | 0 | 0 | 0 | 0 | 0 | 0 | 1.4 | 22 |
| 5 | 1 | 0.4 | 0.32 | 0.06 | 0.6 | 0 | 1.03 | 0 | 0 | 1198 | 24 | 28752 | 0 | 0 | 0 | 10.9 | 38 |
| 6 | 1.02 | 0.4 | 0.31 | 0.06 | 0.59 | 0 | 0.56 | 0 | 0 | 972 | 24 | 23328 | 0 | 0 | 0 | 10.2 | 33 |
| 7 | 1.01 | 0.4 | 0.3 | 0.06 | 0.6 | 0.46 | 0.11 | 0 | 0 | 1053 | 7 | 7371 | 0 | 0 | 0 | 4.4 | 32 |
| 8 | 0.97 | 0.38 | 0.29 | 0.06 | 0.63 | 0 | 0.1 | 0 | 0.49 | 1296 | 4 | 5184 | 0 | 0 | 0 | 5.7 | 34 |
| 9 | 0.97 | 0.35 | 0.26 | 0.06 | 0.66 | 0 | 0.12 | 0 | 0 | 1215 | 24 | 29160 | 1 | 91 | 91 | 10.0 | 48 |
| 10 | 0.98 | 0.38 | 0.1 | 0.06 | 0.63 | 0 | 0.12 | 0 | 0 | 389 | 5 | 1945 | 0 | 0 | 0 | 3.3 | 46 |
| 11 | 0.96 | 0.35 | 0.19 | 0.05 | 0.63 | 0 | 0.12 | 0 | 0 | 972 | 11 | 10692 | 0 | 0 | 0 | 5.0 | 56 |
| 12 | 1.03 | 0.4 | 0.54 | 0.06 | 0.64 | 0 | 0.13 | 0 | 0 | 1053 | 38 | 40014 | 4 | 96 | 384 | 15.9 | 27 |
| 13 | 0.99 | 0.39 | 0.97 | 0.06 | 0.62 | 0 | 0.1 | 0 | 0 | 891 | 39 | 34749 | 14 | 102 | 1428 | 53.3 | 31 |
| 14 | 1.01 | 0.39 | 0.29 | 0.06 | 0.61 | 0 | 0.11 | 0.41 | 0 | 972 | 10 | 9720 | 7 | 104 | 728 | 31.0 | 58 |

As shown in FIG. 2, it is seen that, if no Al—Cu intermetallic compound is generated, the area of filiform rust correlates with Et of the matrix precipitates and tends to increase as Et of the matrix precipitates increases. In other words, by calculating Et of the matrix precipitates through the surface observation of an aluminum sheet and checking this Et against a graph created in advance, it is possible to predict the amount of corrosion due to filiform rust and to evaluate the corrosion resistance of the aluminum sheet.

As shown in FIG. 3, it is seen that, if an Al—Cu intermetallic compound is generated, the area of filiform rust correlates with Et of the Al—Cu-based intermetallic compound and tends to increase as Et of the Al—Cu-based intermetallic compound increases. In other words, by calculating Et of the Al—Cu-based intermetallic compound through the surface observation of an aluminum sheet and checking this Et against a graph created in advance, it is possible to predict the amount of corrosion due to filiform rust and to evaluate the corrosion resistance of the aluminum sheet.

As described above, according to the method for evaluating corrosion resistance of an aluminum sheet in the present embodiment, the potential difference between the dissimilar metal particles and their surroundings on the surface of the aluminum sheet is observed and the product of this potential difference and the number of the dissimilar metal particles is calculated. In this way, it is possible to evaluate the corrosion resistance of an aluminum sheet without performing a long-term corrosion resistance test.

Hereinabove, the embodiment for carrying out the invention is described specifically, but the scope of the present disclosure should not be limited to the above description and should be interpreted broadly based on claims. In addition, the scope of the present invention also includes various alternations and modifications based on the above description.

For example, an observation device other than the Kelvin force microscope may be used to observe the observation surface of the aluminum sheet, as long as the device can observe the potential difference between dissimilar metal particles and their surroundings.

What is claimed is:

1. A method for evaluating corrosion resistance of an aluminum sheet comprising:
preparing an aluminum sheet containing dissimilar metal particles in a matrix metal;
creating an observation surface by treating a surface of the aluminum sheet with hydrochloric acid;
observing a potential difference between the dissimilar metal particles and their surroundings by using an observation device; and
evaluating corrosion resistance of the aluminum sheet from a product of the potential difference and the number of the dissimilar metal particles.

2. The method for evaluating corrosion resistance of an aluminum sheet according to claim 1, wherein a maximum height difference of the observation surface is 200 nm or less.

3. The method for evaluating corrosion resistance of an aluminum sheet according to claim 1, wherein the observation device is a Kelvin force microscope.

4. The method for evaluating corrosion resistance of an aluminum sheet according to claim 1, wherein aluminum used in the aluminum sheet is an Al—Mg—Si-based alloy.

5. The method for evaluating corrosion resistance of an aluminum sheet according to claim 1, wherein
the dissimilar metal particles are at least matrix precipitates or particles of an Al—Cu-based intermetallic compound,
in a case where there is no Al—Cu-based intermetallic compound, the corrosion resistance of the aluminum sheet is evaluated based on the matrix precipitates, and
in a case where there is an Al—Cu-based intermetallic compound, the corrosion resistance of the aluminum sheet is evaluated based on the Al—Cu-based intermetallic compound.
